(19) United States
(12) Patent Application Publication
(10) Pub. No.: US 2010/0074847 A1
(43) Pub. Date: Mar. 25, 2010

(54) Chromophore Probes for Optical Imaging

(76) Inventors: Karen N. Madden, Bedford, MA (US); Kirtland G. Poss, Bedford, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/409,257

(22) Filed: Mar. 23, 2009

Related U.S. Application Data
(63) Continuation of application No. 10/888,950, filed on Jul. 9, 2004, now abandoned.
Application No. 10/888,950 is a continuation of application No. PCT/US03/01346, filed on Jan. 15, 2003.
(60) Provisional application No. 60/349,844, filed on Jan. 16, 2002.

(51) Int. Cl. A61K 49/00; C07K 14/76; C07K 14/79; C12N 5/00

(52) U.S. Cl. 424/9.6; 530/362; 530/394; 435/325

(57) ABSTRACT

Chromophore probes that are capable of being taken up by, retained by or bound to a biocompatible molecule to form an imaging construct are provided. Various activation strategies of the resulting imaging construct are also provided.

### RELATED APPLICATIONS

This application is a Continuation of International Application No. PCT/US03/01346, which designated the United States and was filed on Jan. 15, 2003, published in English, which claims the benefit of U.S. Provisional Application No. 60/349,844, filed on Jan. 16, 2002. The entire teachings of the above applications are incorporated herein by reference.

### FIELD OF THE INVENTION

The invention relates to biochemistry, cell biology, and optical imaging.

### BACKGROUND OF THE INVENTION

Near infrared wavelengths (approx. 600-1000 nm) have been used in optical imaging of internal tissues, because near infrared radiation exhibits tissue penetration of up to 15 centimeters. See, e.g., Wyatt, 1997, “Cerebral oxygenation and haemodynamics in the fetus and newborn infant,” Phil. Trans. R. Soc. London B 352:701-706; and Tromberg et al., 1997, “Non-invasive measurements of breast tissue optical properties using frequency-domain photo migration,” Phil. Trans. R. Soc. London B 352:661-667.

Advantages of near infrared imaging over other currently used clinical imaging techniques include the following: potential for simultaneous use of multiple, distinguishable probes (important in molecular imaging); high temporal resolution (important in functional imaging); high spatial resolution (important in in vivo microscopy); and safety (no ionizing radiation).

In near infrared fluorescence imaging, filtered light or a laser with a defined bandwidth is used as a source of excitation light. The excitation light travels through body tissues. When it encounters a near infrared fluorescent molecule (“contrast agent”), the excitation light is absorbed. The fluorescent molecule then emits light that has detectably different properties (i.e., spectral properties of the probe (slightly longer wavelength), e.g., fluorescence) from the excitation light. Despite good penetration of biological tissues by light, conventional near infrared fluorescence probes are subject to many of the same limitations encountered with other contrast agents, including low target/background ratios.

### SUMMARY OF THE INVENTION

The invention is based on: (1) the design of chromophore probes that are capable of being taken up by, retained by or bound to (either covalently or non-covalently) a biocompatible molecule to form an imaging construct and (2) various activation strategies of the resulting imaging construct, e.g., fluorescence quenching/dequenching, wavelength shifts, polarization, and fluorescence lifetime. The imaging construct is comprised of:

1) A signal or image generating chromophore

2) A chromophore targeting moiety

3) A chromophore attachment moiety

In one aspect, the invention features an imaging construct comprising a chromophore probe and a chromophore targeting moiety that allows the chromophore probe to chemically link to the chromophore attachment moiety and to be maintained in a spectral property altering state, so that upon activation of the resulting imaging construct, the optical properties of the chromophore are altered.

A “chromophore” includes, but is not limited to, a fluorochrome, a non-fluorochrome chromophore, a fluorescence quencher, an absorption chromophore, a fluorophore, any organic or inorganic dye, metal chelate, or any fluorescent enzyme substrate.

A “chromophore targeting moiety” is any molecule or structural feature that allows the chromophore probe to chemically link to the chromophore attachment moiety.

A “chromophore attachment moiety” is a biocompatible molecule, to which one or more chromophores can be chemically linked and maintained in a spectral property altering state. Endogenous biomolecules are preferred and include, but are not limited to, albumin, transferrin, fatty acid binding proteins, globulins, red blood cells, lymphocytes, stem cells, antibodies and lipoproteins.

“Chemically linked” is meant connected by any attractive force between atoms strong enough to allow the combined aggregate to function as a unit. This includes, but is not limited to, chemical bonds such as covalent bonds (e.g., polar or nonpolar), non-covalent bonds such as ionic bonds, metallic bonds, and bridge bonds, and hydrophobic interactions and van der Waals interactions.

“Spectral property altering state” is the state of one or more chromophores that permits them to interact photochemically with one another, or with structural elements within the chromophore, chromophore targeting moiety or chromophore attachment moiety such that the detectable signal of the chromophores are altered when compared to the activated state. Such altering of detectable signal includes, but is not limited to, fluorescence quenching/dequenching, wavelength shifts, polarization, and fluorescence lifetime changes.

By “activation” is meant any change that alters a detectable property, e.g., an optical property, of the imaging construct or chromophore probe. This includes, but is not limited to, any modification, alteration or binding (covalent or non-covalent) of the construct or chromophore probe that results in a detectable difference in properties, e.g., optical properties of the chromophore, e.g., changes in the fluorescence signal amplitude (e.g., dequenching and quenching), change in wavelength, fluorescence lifetime, spectral properties, or polarity. Activation can be, without limitation, by enzymatic or pH mediated cleavage, enzymatic conversion, phosphorylation or dephosphorylation, analyte binding such as association with, H⁺, Na⁺, K⁺, Ca²⁺, Cl⁻, or another analyte, any chemical modification of the chromophore, or natural release of the chromophore (i.e., equilibrium).

The invention also features in vivo optical imaging methods. In one embodiment the method includes the steps of: (a) administering to a subject a chromophore probe with a chromophore targeting moiety; (b) allowing the chromophore probe to chemically link to the chromophore attachment moiety via the chromophore targeting moiety and be maintained in a spectral property altering state; (c) allowing time for molecules in the target tissue to activate the resulting imaging construct; (d) illuminating the target tissue with light of a wavelength absorbable by the chromophore; and (e) detecting the optical signal emitted by the chromophore.

These steps can also be repeated at predetermined intervals thereby allowing for the evaluation of emitted signal of the chromophores in a subject over time. The emitted signal may take the form of an image. The subject may be a mammal, including a human, as well as other experimental animal models such as xenopus, zebrafish, and C. elegans.

The invention also features an in vivo method for selectively detecting and imaging two or more chromophores probes simultaneously. The method includes administering to a subject two or more chromophore probes, whose optical properties are distinguishable from that of the other. The method therefore, allows the recording of multiple events or targets.

The methods of the invention can be used to determine a number of indicia, including tracking the localization of the imaging construct in the subject over time and assessing changes in the level of the imaging construct in the subject over time. The methods of the invention can also be used in the detection, characterization and/or determination of the localization of a disease, the severity of a disease or a disease—associated condition, and monitoring and guiding various therapeutic interventions, such as surgical procedures and monitoring drug therapy. Examples of such disease or disease-conditions include inflammation (e.g., inflammation caused by arthritis, for example, rheumatoid arthritis), all types of cancer, cardiovascular disease (e.g., atherosclerosis and inflammatory conditions of blood vessels), dermatologic disease (e.g., Kaposi's Sarcoma, psoriasis), ophthalmic disease (e.g., macular degeneration, diabetic retinopathy), infectious disease, immunologic disease (e.g., Acquired Immunodeficiency Syndrome, lymphoma and multiple sclerosis), neurodegenerative disease (e.g., Alzheimer's disease), and bone-related disease (e.g., osteoporosis and primary and metastatic bone tumors). The methods of the invention can therefore be used, for example, to determine the presence of tumor cells and localization of tumor cells, the presence and localization of inflammation, the presence and localization of vascular disease including areas at risk for acute occlusion (vulnerable plaques) in coronary and peripheral arteries and regions of expanding aneurysms, as well as unstable plaque in carotid arteries.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention is based on: (1) the design of chromophore probes that are capable of being taken up by, retained by or bound to (either covalently or non-covalently) to a biocompatible molecule to form an imaging construct and (2) various activation strategies of the resulting imaging construct, e.g., fluorescence quenching/dequenching, wavelength shifts, polarization, and fluorescence lifetime. The imaging construct is comprised of:

1) A signal or image generating chromophore

2) A chromophore targeting moiety

3) A chromophore attachment moiety

### Signal or Image Generating Chromophore

Chromophores with excitation and emission wavelengths in the red and near infrared spectrum are preferred, i.e., 550-1300 nm. Use of this portion of the electromagnetic spectrum maximizes tissue penetration and minimizes absorption by physiologically abundant absorbers such as hemoglobin (<650 nm) and water (>1200 nm). Ideal near infrared chromophores for in vivo use exhibit the following characteristics: (1) narrow spectral characteristics, (2) high sensitivity (quantum yield), (3) biocompatibility, and (4) decoupled absorption and excitation spectra.

Various near infrared chromophores are commercially available and can be used to construct probes according to this invention. Exemplary chromophores include the following: Cy5.5, Cy5 and Cy7 (Amersham, Arlington Hts., IL); IRD41 and IRD700 (LI-COR, Lincoln, Nebr.); NIR-1 and 105-OSu, (Dejindo, Kumamoto, Japan); Alexflour 660, Alexflour 680 (Molecular Probes, Eugene, Oreg.), LaJolla Blue (Diatron, Miami, Fla.); FAR-Blue, FAR-Green One, and FAR-Green Two (Innosense, Giacosa, Italy), ADS 790-NS and ADS 821-NS (American Dye Source, Montreal, Canada), indocyanine green (ICG) and its analogs (Licha, et al., 1996, SPIE 2927:192-198; Ito et al., U.S. Pat. No. 5,968,479); indotricarbocyanine (ITC; WO 98/47538); fluorescent quantum dots (zinc sulfide-capped cadmium selenide nanocrystals) (QuantumDot Corporation; www.qdots.com) and chelated lanthanide compounds. Fluorescent lanthanide metals include europium and terbium. Fluorescence properties of lanthanides are described in Lackowicz, 1999, Principles of Fluorescence Spectroscopy, 2^{nd} Ed., Kluwar Academic, New York.

Table I summarizes information on the properties of several exemplary near infrared chromophores that could be used in the present invention.

TABLE I
Exemplary Near Infrared Chromophores

| | | | | | Extinction |
|---|---|---|---|---|---|
| | | λex | λem | MW | Coefficient |
| Chromophore | Supplier | (nm) | (nm) | (gmol⁻¹) | (M⁻¹cm⁻¹) |
| Cy5.5 | Amersharm | 675 | 694 | 1128.41 | 250,000 |
| Far-Blue | Medway | 660 | 678 | 825 | 150,000 |
| Far-Green | Medway | 800 | 820 | 992 | 150,000 |
| ADS 790 NS | American Dye | 791 | >791 | 824.07 | Unknown |
| | Source | | | | |
| ADS 821 NS | American Dye | 820 | >820 | 924.07 | Unknown |
| | Source | | | | |
| Alex Fluor 647 | Molecular | 650 | 668 | 1300 | 239,000 |
| | Probes | | | | |
| Alex Fluor 660 | Molecular | 663 | 690 | 1100 | 132,000 |
| | Probes | | | | |
| Alex Fluor 680 | Molecular | 679 | 702 | 1150 | 184,000 |
| | Probes | | | | |
| IC5-OSu | Dojindo | 641 | 657 | 630.23 | Unknown |

Although red and near infrared chromophores are preferred, it will be appreciated that the use of chromophores with excitation and emission wavelengths in other spectrums, such as the visible and ultraviolet light spectrum, can also be employed in the compositions and methods of the present invention.

Fluorescent enzyme substrates such as those described in U.S. Pat. Nos. 5,605,809 and 6,248,904, and commercially sold by Molecular Probes (Eugene, Oreg.), can also be used as the signal or image generating chromophore with the present invention. Such fluorescent enzyme substrates can be activated by a number of different mechanisms, including but not limited to enzymatic or pH mediated cleavage, phosphorylation or dephosphorylation.

In addition, a number of fluorescent chromophores are known in the art that have a high and selective affinity for albumin in vitro and in vivo (U.S. Pat. No. 5,073,171; Williams et al. (1993) Anal. Chem. 65:601-605) that could also be used in the present invention.

### Chromophore Targeting Moiety

For targeting chromophore binding to the chromophore attachment moiety, a wide range of chromophore targeting moieties and strategies can be used, depending on the nature of the chromophore attachment moiety. For example, for targeting chromophore binding to albumin, a wide range of hydrophobic and amphiphilic chromophore targeting moieties can be used including aliphatic or aryl groups, nitrogens, oxygens, sulfurs, halogens, alkyl groups, amides, esters and sulfonamides (Kragh-Hansen (1981) Pharm. Rev. 33:17-53; He et al. (1992) 358:209-215; Carter (Adv. Protein Chem. (1994) 45:153-203). For binding to albumin, it is preferred to have negatively charged molecules or molecules containing negatively charged oxygens, or sulfurs or fluorines, or molecules of net neutral charge. For binding to alpha acid glycoprotein, it is preferred to have at least some portion of the chromophore targeting moiety be positively charged. For binding to globulins, some portion of the chromophore targeting moiety could be steroidal in nature and for lipoproteins, some portion of the chromophore targeting moiety could be lipophilic or fatty-acid like.

Alternatively, the chromophore probe can be covalently linked to the chromophore attachment moiety using any suitable reactive group as the chromophore targeting moiety and a compatible functional group on the chromophore attachment moiety or spacer. For example, a carboxyl group (or activated ester) as the chromophore targeting moiety can be used to form an amide linkage with a primary amine such as the E-amino group of the lysyl side chain on the chromophore attachment moiety. Alternatively, a thiol or disulfide group can be used as the chromophore targeting moiety that is capable of reacting with a thiol or disulfide group on the chromophore attachment moiety, such as cysteine residue. A particular thiol binding group on human serum albumin is cysteine 34.

Peptides, peptide mimetics, glycoproteins, carbohydrates, antibodies, and fragments thereof can also be used as chromophore targeting moieties to target chromophore binding to the chromophore attachment moiety.

Techniques for targeting radioactive isotopes and paramagnetic contrast agents to circulating cells such as red blood cells and lymphocytes are well known in the art (U.S. Pat. No. 5,277,892; U.S. Pat. No. 4,935,223; U.S. Pat. No. 5,116,597; U.S. Pat. No. 6,146,614) and can also be used in the design and construction of the chromophore targeting moiety to target chromophore binding to such circulating cells. Such techniques include, but are not limited to, electroporation of cells ex vivo which involves the exposure of cells to a pulsed electric field to cause the formation of pores in the cell membrane that allow transfer of molecules into the cell. Alternatively, anti-CD4 antibodies can be used to target CD4 positive lymphocytes.

In addition, chemoinformatic methods may also be used to design and predict binding affinities of chromophore probes and chromophore targeting moieties to various chromophore attachment moieties (Colmenarejo et al. (2001) J. Med. Chem. 44:4370-4378).

### Chromophore Attachment Moiety

A chromophore attachment moiety can be any biocompatible molecule that allows one or more chromophores to be linked thereto. Preferred biocompatible molecules are endogenous native biomolecules. Because albumin is a very small and abundant plasma protein (MW 69,000), and functions to transport molecules from the vasculature into cells, it is a preferred endogenous chromophore attachment moiety. In addition, other endogenous biological molecules include, but are not limited to, antibodies, such as IgM and IgG, transferrin, fatty acid binding proteins, globulins, lipoproteins, red blood cells, lymphocytes, platelets, endothelial cells, stem cells, p90, p38, and any cellular receptor.

It is well known in the art that certain chromophore attachment moieties such as albumin and transferrin, accumulate in solid tumors and can be used as carriers for the delivery of imaging and therapeutic agents to tumors and sites of inflammation (Becker et al. (2000) Photochem. Photobiol. 72:234-241; Kremer et al. (2000); 22:481-489; Schilling et al. (1992) 19:685-695; Nucl. Med. Biol. (2001) 28:895-902; Brasseur et al. (1999) Photochem. Photobiol. 69:345-352; Gatter et al. (1983) J. Clin. Path. 36:539-545; Hamlin & Newman (1994) 26:45-56; Rennen et al. (2001) 28:401-408). This is due, in part, to the high density and increased permeability of the vasculature within many tumors and sites of inflammation. (Matsumura & Maeda (1986) Cancer Res. 46:6387-6392). Therefore in many pathologic conditions such as tumors, inflammation, and arteriosclerotic plaques, where the capillaries are “leaky”, there are high local concentrations of albumin.

By virtue of this accumulation, the imaging constructs of this invention can be used to image tumor tissues and sites of inflammation, even if the enzyme(s) activating the novel construct are not entirely disease specific. The methods of the invention can therefore be used, for example, to determine the presence of tumor cells and localization of tumor cells, the presence and localization of inflammation, the presence and localization of vascular disease including areas at risk for acute occlusion (vulnerable plaques) in coronary and peripheral arteries and regions of expanding aneurysms. Alternatively, this accumulation can also be exploited to deliver specific fluorogenic enzyme substrates to interrogate for relatively more disease specific enzymes.

In one embodiment, the invention features an imaging construct comprising a chromophore probe that has been designed to chemically link to an endogenous biocompatible molecule. The chromophore probe is administered to the subject and binding of the chromophore to the chromophore attachment moiety occurs in situ in vivo. As a result of chemically linking to the chromophore attachment moiety, the chromophore probe takes on the biological properties of the endogenous chromophore attachment moiety, including the half-life. After circulation of the resulting imaging construct and allowing time for molecules in the target tissue to activate the construct, the optical signal emitted by the chromophore is detected.

In a preferred embodiment, the endogenous chromophore attachment moiety is albumin and activation of the imaging construct occurs via degradation of albumin by molecules present in the target tissue. Although fibroblasts in peripheral tissues are the primary sites of albumin degradation, albumin is also capable of being degraded by almost every organ of the body. Specifically, albumin is catabolized extensively by tumors and inflammatory cells and therefore this preferred embodiment can be used to image tumors and sites of inflammation. Some of the enzymes responsible for albumin degradation include cathepsins.

In another embodiment, the chromophore probe is a fluorescent enzyme substrate that has been designed to chemically link to the chromophore attachment moiety and activation occurs via enzymatic or pH mediated cleavage, or phosphorylation or dephosphorylation of the fluorescent enzyme substrate. Such fluorescent enzyme substrates include, but are not limited to those described in U.S. Pat. Nos. 5,605,809 and 6,248,904, and those commercially sold by Molecular Probes (Eugene, Oreg.).

In a preferred embodiment, the chromophores are intramolecularly quenched. Several mechanisms are known including resonance energy transfer between two chromophores. In this mechanism, the emission spectrum of a first chromophore should be very similar to the excitation of a second chromophore, which is in close proximity to the first chromophore. Efficiency of energy transfer is inversely proportional to r⁶, where r is the distance between the quenched chromophore and excited chromophore. Self-quenching can also result from chromophore aggregation or excimer formation. This effect is strictly concentration dependent. Quenching also can result from a non-polar-to-polar environmental change.

In another embodiment, the chromophore probe may be pre-bound (using any of the chromophore binding moieties and strategies previously described) to the chromophore attachment moiety ex vivo. For example, the chromophore probe may be mixed with sterile albumin or plasma replacement solution and the resulting imaging probe construct injected into the subject. Alternatively, blood may be drawn from the subject and the chromophore probe can be mixed with the subject's blood and the resulting imaging construct re-injected into the subject. After circulation of the resulting imaging construct and allowing time for molecules in the target tissue to activate the construct, the optical signal emitted by the chromophore is detected.

### In Vivo Optical Imaging

Although the invention involves novel chromophore probes, general principles of fluorescence, optical image acquisition, and image processing can be applied in the practice of the invention. For a review of optical imaging techniques, see, e.g., Alfano et al., 1997, “Advances in Optical Imaging of Biomedical Media,” Ann. NY Acad. Sci., 820:248-270.

An imaging system useful in the practice of this invention typically includes three basic components: (1) an appropriate light source for chromophore excitation, (2) a means for separating or distinguishing emissions from light used for chromophore excitation, and (3) a detection system.

Preferably, the light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., 1994, Proc. Natl. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418. Information on near infrared lasers for imaging can be found at http://www.imds.com and various other well-known sources.

A high pass or bandpass filter (700 nm) can be used to separate optical emissions from excitation light. A suitable high pass or bandpass filter is commercially available from Omega Optical.

In general, the light detection system can be viewed as including a light gathering/image forming component and a light detection/image recording component. Although the light detection system may be a single integrated device that incorporates both components, the light gathering/image forming component and light detection/image recording component will be discussed separately.

A particularly useful light gathering/image forming component is an endoscope. Endoscopic devices and techniques which have been used for in vivo optical imaging of numerous tissues and organs, including peritoneum (Gahlen et al., 1999, J. Photochem. Photobiol. B 52:131-135), ovarian cancer (Major et al., 1997, Gynecol. Oncol. 66:122-132), colon (Mycek et al., 1998, Gastrointest. Endosc. 48:390-394; Stepp et al., 1998, Endoscopy 30:379-386) bile ducts (Izuishi et al., 1999, Hepatogastroenterology 46:804-807), stomach (Abe et al., 2000, Endoscopy 32:281-286), bladder (Kriegmair et al., 1999, Urol. Int. 63:27-31; Riedl et al., 1999, J. Endourol. 13:755-759), and brain (Ward, 1998, J. Laser Appl. 10:224-228) can be employed in the practice of the present invention.

Other types of light gathering components useful in the invention are catheter based devices, including fiber optics devices. Such devices are particularly suitable for intravascular imaging. See, e.g., Tearney et al., 1997, Science 276:2037-2039; Proc. Natl. Acad. Sci. USA 94:4256-4261.

Still other imaging technologies, including phased array technology (Boas et al., 1994, Proc. Natl. Acad. Sci. USA 91:4887-4891; Chance, 1998, Ann. NY Acad. Sci. 838:29-45), diffuse optical tomography (Cheng et al., 1998, Optics Express 3:118-123; Siegel et al., 1999, Optics Express 4:287-298), intravital microscopy (Dellian et al., 2000, Br. J. Cancer 82:1513-1518; Monsky et al., 1999, Cancer Res. 59:4129-4135; Fukumura et al., 1998, Cell 94:715-725), and confocal imaging (Korlach et al., 1999, Proc. Natl. Acad. Sci. USA 96:8461-8466; Rajadhyaksha et al., 1995, J. Invest. Dermatol. 104:946-952; Gonzalez et al., 1999, J. Med. 30:337-356) can be employed in the practice of the present invention.

Any suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a near infrared imaging system, and operating the system is within ordinary skill in the art.

It will be appreciated that the compositions and methods of the present invention may be used in combination with other imaging compositions and methods. For example, the methods of the present invention may be used in combination with traditional imaging modalities such as CT, PET, SPECT, MRI, and such probes may contain components, such as iodine, gadolinium atoms and radioactive isotopes, which change imaging characteristics of tissues when imaged using CT, PET, SPECT, and MR.

1. An imaging construct comprising a chromophore probe and a chromophore targeting moiety that allows the chromophore probe to chemically link to a chromophore attachment moiety and to be maintained in a spectral property altering state, so that upon activation of the resulting imaging construct, the optical properties of the chromophore are altered.

2. The imaging construct of claim 1, wherein the imaging construct is activated by:
(a) enzymatic cleavage;
(b) pH mediated cleavage;
(c) phosphorylation;
(d) dephosphorylation;
(e) conformation change;
(f) analyte binding;
(g) chemical modification of the chromophore; or
(h) receptor binding.

3. The imaging construct of claim 1, wherein the imaging construct is activated by enzymatic cleavage of the chromophore attachment moiety.

4. The imaging construct of claim 1, wherein the chromophores are red to near-infrared fluorochromes with excitation and emission wavelengths in the range of 550 to 1300 nm.

5. The imaging construct of claim 1, wherein the chromophore is covalently linked to the chromophore attachment moiety.

6. The imaging construct of claim 1, wherein the chromophore is non-covalently linked to the chromophore attachment moiety.

7. The imaging construct of claim 1, wherein the chromophore attachment moiety is endogenous.

8. The imaging construct of claim 1, wherein the chromophore attachment moiety is albumin.

9. The imaging construct of claim 1, wherein the chromophore attachment moiety is transferrin.

10. The imaging construct of claim 1, wherein the chromophore attachment moiety is red blood cells.

11. The imaging construct of claim 1, wherein the chromophore attachment moiety is a lymphocyte.

12. The imaging construct of claim 1, wherein the chromophore attachment moiety is a stem cell.

13. A method of in vivo optical imaging, the method comprising:
(a) administering to a subject a chromophore probe with a chromophore targeting moiety;
(b) allowing the chromophore probe to chemically link to the chromophore attachment moiety and be maintained in a spectral property altering state;
(c) allowing time for molecules in a target tissue to activate the resulting imaging construct;
(d) illuminating the target tissue with light of a wavelength absorbable by the chromophore; and
(e) detecting the optical signal emitted by the chromophore.

14. A method of in vivo optical imaging, the method comprising:
(a) withdrawing a sample of a subject's blood;
(b) mixing the subject's blood (or any component thereof) with a chromophore probe and allowing the chromophore probe to chemically link to a chromophore attachment moiety and be maintained in a spectral property altering state;
(c) injecting the resulting imaging construct back into the subject;
(d) allowing adequate time for the imaging construct to be activated within a target tissue;
(e) illuminating the target tissue with light of a wavelength absorbable by the chromophores; and
(f) detecting the signal emitted by the chromophores.

15. The method of claim 13, wherein steps (a)-(e) are repeated at predetermined intervals thereby allowing for evaluation of emitted signal of the chromophores in the subject over time.

16. The method of claim 13, wherein the signal emitted by the chromophores is used to construct an image.

17. The method of claim 13, wherein the subject is a mammal.

18. The method of claim 13, wherein the subject is a human.

19. The method of claim 13, wherein the illuminating and detecting steps are done using an endoscope, catheter, tomographic systems (including diffuse optical tomography), surgical goggles with attached bandpass filters, or intraoperative microscope.

20. The method of claim 13, wherein the method is used in detection of a disease.

21. The method of claim 13, wherein the method is used in monitoring or dictating a therapeutic course of action for a treatment of a disease.

22. The method of claim 20, wherein the disease is selected from the group consisting of cancer, cardiovascular diseases, neurodegenerative diseases, immunologic diseases, autoimmune diseases, inherited diseases, infectious diseases, bone diseases, and environmental diseases.
